(19) United States
(12) Patent Application Publication
(10) Pub. No.: US 2012/0165908 A1
(43) Pub. Date: Jun. 28, 2012

(54) Cold/hot therapy device with temperature control

(75) Inventors: Hong-Sen Kou, Taipei City (TW); Ji-Jen Lee, Taipei City (TW); Chun-Che Hsieh, New Taipei City (TW); Bo-Min Shih, Luzhu Township (TW); Hsueh-Erh Liu, Taipei City (TW)

(73) Assignees: Tatung Company, Taipei (TW); Tatung University, Taipei (TW)

(21) Appl. No.: 13/064,164

(22) Filed: Mar. 9, 2011

(30) Foreign Application Priority Data
Dec. 28, 2010 (TW) 099146393

(51) Int. Cl. A61F 7/10

(52) U.S. Cl. 607/104

(57) ABSTRACT

A cold/hot therapy device with temperature control includes a housing, a pump, a cooling chip, a cooling head, a cooling fin, and a bag. The pump, the cooling chip and the cooling head are fixedly arranged in the accommodating chamber of the housing. The cooling chip includes a cool-effecting surface and a cooling surface. The cooling head is adjacent to the cool-effecting surface, and includes a water inlet and a water outlet, where the water inlet communicates with the pump outlet. The cooling fin is fixedly arranged on the cooling surface of the cooling chip, and is positioned in the hollow passage of the housing. The bag includes an inlet tube and an outlet tube, where the inlet tube communicates with the water outlet of the cooling head, and the outlet tube communicates with the pump inlet. Accordingly, a cold or hot therapy function can be achieved.

### BACKGROUND OF THE INVENTION

1. Field of the Invention

The present invention relates to a cold/hot therapy device with temperature control, and more particularly, to a cold/hot therapy device with temperature control adapted for family personal and hospital health care.

2. Description of Related Art

A well-known cold therapy device relates to a cold bag filled with cold water or ice cubes and then laid on a certain part of a users' body for keeping cool of that part of the body. However, such a cold bag requires human efforts to observe continuously whether the temperature of the water in the cold bag has reached to a level of thermal balance, namely whether the temperature of the water in the cold bag has been equal to room temperature such that a function of cold therapy may fail. Suppose the temperature of the cold bag loses its function in lowering the temperature at the certain part of the user's body, the healthcare personnel will need to replace or fill in icy water or ice cubes. Namely, the known cold bag, when starting to use, may incur injuries such as frostbite or nerve palsy; while after a certain time of use, replacing or filling icy water or ice cubes is necessary, significantly increasing the workload of healthcare personnel.

It is found that cold therapy devices, using cooling chips with cooling characteristic, have been developed. Referring to FIGS. 1 and 2, exploded and perspective views illustrating a conventional cold therapy device, in the cold therapy device a housing 70 has an internal space 72 in which a reservoir 80, a cooling fin 86, a cooling fan 82, and a power supply 75 are fixedly arranged. A plurality of vents 71 are provided at circumference of the housing 70 and near the cooling fin 86. A cold bag 95 has an inlet tube 951 in communication with an outlet 882 of a pump 88, while an outlet tube 952 is in communication with the reservoir 80. Therefore, coolant 81 flows out of the outlet 882 of the pump 88 and into the cold bag 95 through the inlet tube 951, then flows back to the reservoir 80 through the outlet tube 952, and enters into an inlet 881 of the pump 88. This will form a closed circulation loop.

A cover 90 is provided with two ports 901,902 for passing through the outlet and inlet tubes 952,951 of the cold bag 95. A cooling chip 85 has a cooling surface 852 on which the cooling fin 86 is arranged, and that the cooling fan 82 is fixedly arranged, toward the cooling fin 86, in the internal space 72 of the housing 70. A cool-effecting surface 851 of the cooling chip 85 provides a cooling characteristic and transfers the same to the coolant 81 in the reservoir 80 so as to lower temperature of the coolant 81, where the coolant 81 is conveyed, by the pump 88, to the cold bag 95. Thus a cold therapy function can be achieved. Thereafter, the coolant 81, which becomes relatively high temperature after the cold therapy, flows back to the reservoir 80 for heat exchange. As such, the coolant 81 can keep, automatically, the cold bag 95 at a state of low temperature.

Given the above, it is understood that the conventional cold therapy device is bulky in dimension, and due to the structural arrangement, the cooling fan has a poor cooling effect. This results in an unsatisfactory cooling effect for the cooling chip, making an undesirable cold therapy function. Moreover, the conventional art can only provide a cold therapy function, but cannot provide a hot therapy function as well. Accordingly, there is still room for improvement for the conventional art.

It is, therefore, significant to solve the problem, as mentioned above, and as such, a “cold/hot therapy device with temperature control” is accomplished after research and persistent experiments.

### SUMMARY OF THE INVENTION

An object of the present invention is to provide a cold/hot therapy device with temperature control, so that cooling effect of the cooling fan can be enhanced through an improved structural arrangement. This will also raise efficiency of the cooling chip and make the overall cold/hot therapy device a more compact dimension and energy saving.

Another object of the present invention is to provide a cold/hot therapy device with temperature control, where not only a cold therapy function can be provided, but also a hot therapy function can be achieved.

To achieve the objects, as mentioned above, the cold/hot therapy device with temperature control comprises a housing, a pump, a cooling chip, a cooling head, a cooling fin, and a bag. The housing includes an accommodating chamber and a hollow passage. The pump is fixedly arranged in the accommodating chamber of the housing, and includes a pump inlet and a pump outlet. The cooling chip is also fixedly arranged in the accommodating chamber of the housing, and includes a cool-effecting surface and a cooling surface. The cooling head is as well fixedly arranged in the accommodating chamber of the housing, and is adjacent to the cool-effecting surface of the cooling chip, and includes a water inlet and a water outlet, where the water inlet communicates with the pump outlet. The cooling fin is fixedly arranged on the cooling surface of the cooling chip, and is positioned in the hollow passage of the housing. The bag includes an inlet tube and an outlet tube, where the inlet tube communicates with the water outlet of the cooling head, and the outlet tube communicates with the pump inlet of the pump.

The cold/hot therapy device, according to the present invention, may further comprise a cooling fan which is fixedly arranged in the hollow passage and faces toward the cooling fin, so that heat exchange effect of the cooling fin can be enhanced and so as the function of the cooling chip.

The cold/hot therapy device, according to the present invention, may further comprise a temperature sensor, which is fixed on the cooling head, for detecting the temperature of the water at that location and to provide water temperature information for the user.

The cold/hot therapy device, according to the present invention, may further comprise a temperature display, which is fixed on the housing, for displaying the water temperature being detected. The temperature display may relate to a liquid crystal display (LCD), or other equivalents.

The cold/hot therapy device, according to the present invention, may further comprise a controller, fixedly arranged in the accommodating chamber of the housing, and a temperature setting device, fixedly arranged on the housing, where the controller is electrically connected with the temperature setting device and the temperature sensor. The temperature setting device can set a target temperature value, while the controller can compare the target temperature value with the temperature detected by the temperature sensor so as to control at least one of the following group: On/Off of the pump and On/Off of the cooling chip. In other words, the controller can control the pump whether to run continuously so as to deliver or not to deliver the water in the cooling head to the bag; or alternatively, to control the cooling chip whether to run continuously so as to absorb or not to absorb the heat from the cooling head and to make the water in the bag comply with the target temperature value.

The controller may further include a reversible circuit for controlling the cooling chip so as to provide a cold or hot therapy function.

According to the present invention, the cooling head may include a channel body and a cooling surface, wherein the channel body is constituted, in series, by a plurality of channels which are parallel from one another, and wherein the depth and the width of the channel are in a proportion of 3 to 4. The channel body is formed zigzag so as to increase contact areas with the cooling surface, and as such, heat exchange effect can be enhanced, making water temperature reaches rapidly to a preset target value.

The housing may consist of a base and a cover. The cover has a plurality of engaging tabs each having a through hole; while the base has a plurality of screw holes corresponding to the through holes, thus the cover and the base can be threadedly engaged together by a plurality of bolts.

Further, according to the present invention, the cover may be provided with a handle such that the cold/hot therapy device can be portable and is easy to be carried.

Still further, according to the present invention, the hollow passage may pass transversely through the housing so as to form an air guide, making forced airflow of the cooling fan flow through the hollow passage without obstruction, and increasing heat exchange effect of the cooling fin and enhancing the function of the cooling chip.

Other objects, advantages, and novel features of the present invention will become more apparent from the following detailed descriptions when taken in conjunction with the accompanying drawings.

### BRIEF DESCRIPTION OF THE DRAWINGS

FIG. 1 is an exploded view illustrating a conventional cold therapy device;

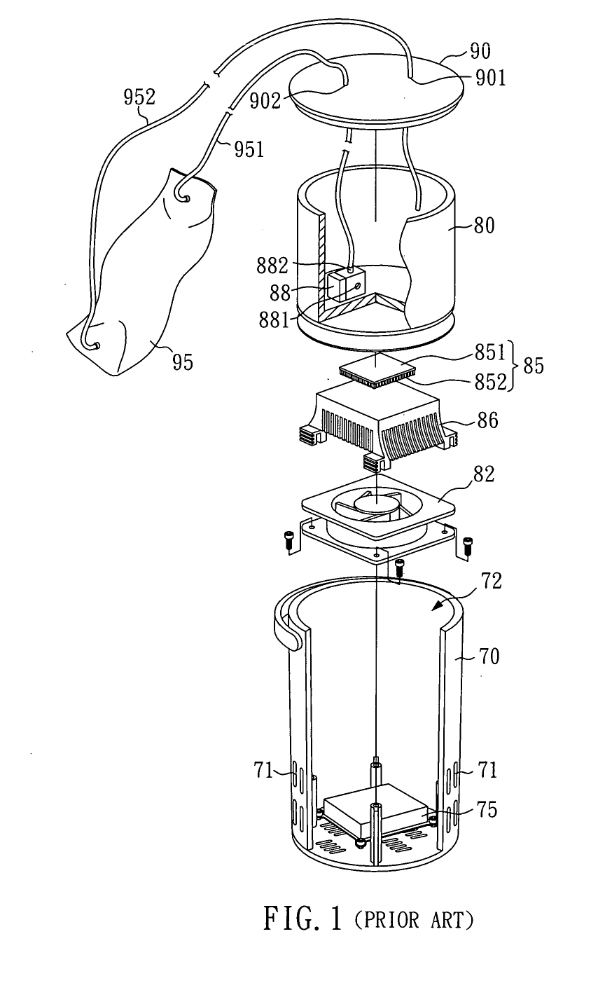

FIG. 2 is a perspective view illustrating the conventional cold therapy device;

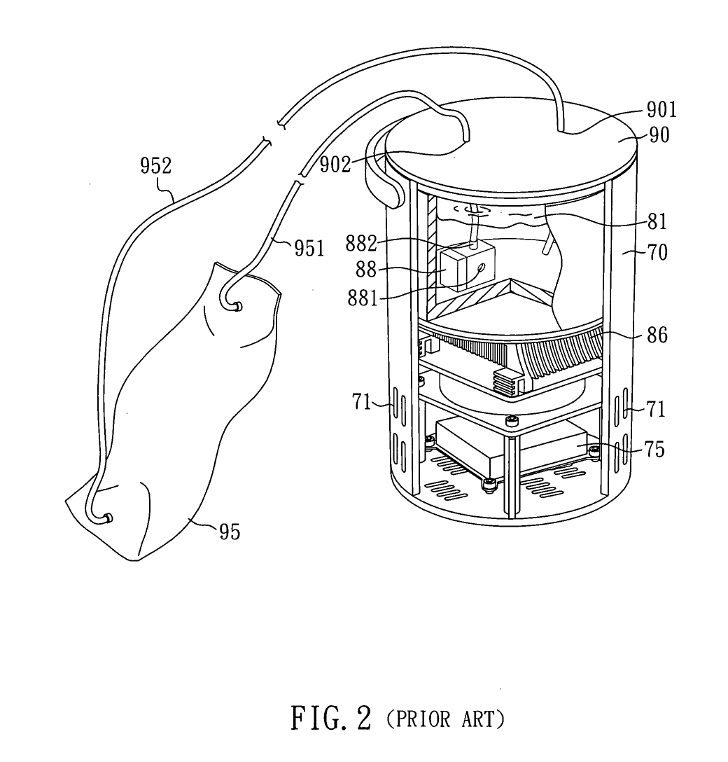

FIG. 3 is an exploded view illustrating a cold/hot therapy device with temperature control according to the present invention;

FIG. 4 is a perspective view illustrating the cold/hot therapy device with temperature control according to the present invention;

FIG. 5 is a block diagram illustrating temperature control of the cold/hot therapy device according to the present invention;

FIG. 6 is a perspective view illustrating a cooling head according to the present invention; and

FIG. 7 is a diagram illustrating a reversible circuit according to the present invention.

### DETAILED DESCRIPTION OF THE PREFERRED EMBODIMENTS

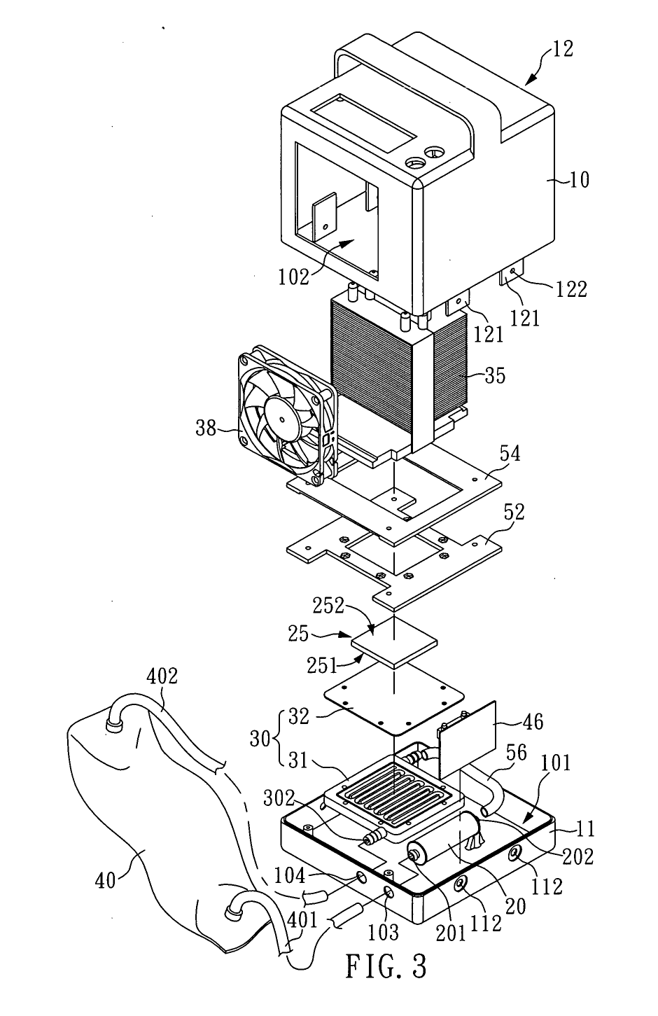

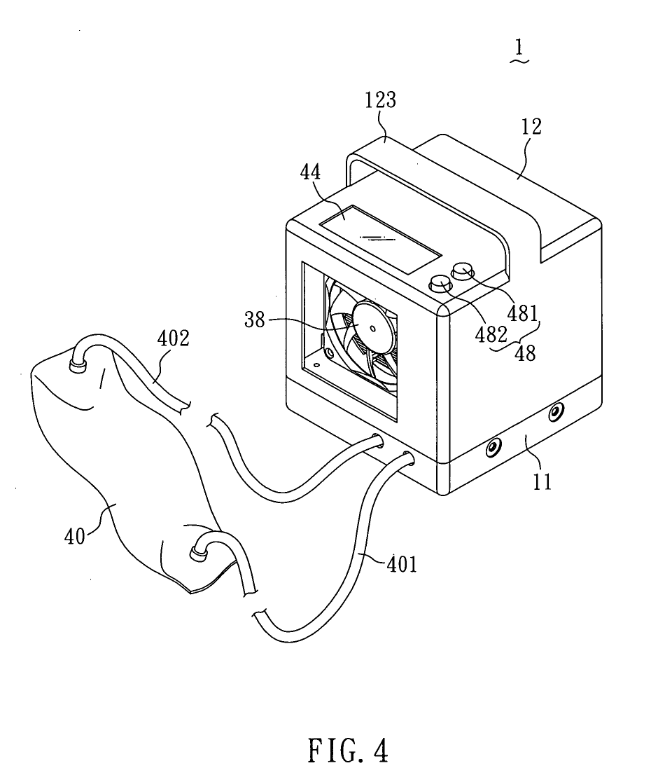

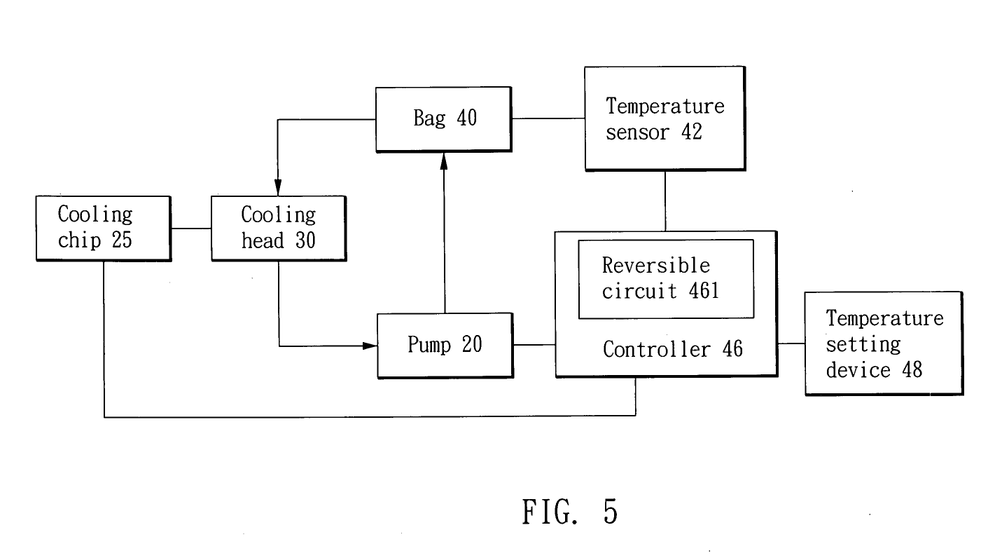

Referring to FIG. 3, an exploded view illustrating a cold/hot therapy device with temperature control according to the present invention, to FIG. 4, a perspective view illustrating the cold/hot therapy device with temperature, and to FIG. 5, a block diagram illustrating temperature control of the cold/hot therapy device, the cold/hot therapy device with temperature comprises a housing 10, a pump 20, a cooling chip (i.e. Thermoelectric Cooling Module) 25, a cooling head 30, a cooling fin 35, a cooling fan 38, a temperature sensor 42, a temperature display 44, a controller 46, a temperature setting device 48, a chip holder 52, a fan holder 54, and a bag 40.

As shown, the housing 10 includes an accommodating chamber 101, a hollow passage 102, an inlet 103, and an outlet 104, wherein the hollow passage 102 passes through the housing 10 transversely. The housing 10 consists of a base 11 and a cover 12. The cover 12 has a plurality of engaging tabs 121 each having a through hole 122; while the base 11 has a plurality of screw holes 112 corresponding to the through holes 122, thus the cover 12 and the base 11 can be threadedly engaged together by a plurality of bolts (not shown). In the present invention, the cover 12 is provided with a handle 123 such that the cold/hot therapy device 1 can be portable and is easy to be carried.

According to the present invention, the pump 20, the cooling chip 25, and the cooling head 30 are all fixedly arranged in the accommodating chamber 101 of the housing 10. The pump 20 includes a pump inlet 201 and a pump outlet 202. The cooling chip 25 includes a cool-effecting surface 251 and a cooling surface 252. The cooling head 30, adjacent to the cool-effecting surface 251 of the cooling chip 25, includes a water inlet 301 and a water outlet 302, where the water inlet 301 communicates with the pump outlet 202 through a tube 56. In the present invention, the cooling chip 25 is fixedly arranged at the cooling head 30 by means of the chip holder 52.

Further, according to the present invention, the cooling fin 35 is fixedly arranged on the cooling surface 252 of the cooling chip 25, and is positioned in the hollow passage 102 of the housing 10. The cooling fan 38 is also fixedly arranged in the hollow passage 102 and faces toward the cooling fin 35. Since forced airflow of the cooling fan 38 flows through the hollow passage 102 without obstruction, heat exchange effect toward the cooling fin 35 can thus be enhanced and so the function of the cooling chip 25. In the present invention, the cooling fan 38 is fixed on the chip holder 52 through the fan holder 54.

The bag 40 includes an inlet tube 402 and an outlet tube 401. The inlet tube 402 communicates with the water outlet 302 of the cooling head 30 through the outlet 104 of the housing 10. The outlet tube 401 communicates with the pump inlet 201 of the pump 20 through the inlet 103 of the housing 10. The cold/hot therapy device 1, according to the present invention, forms itself a sealed interior circulation loop.

As shown in FIG. 5, and also in FIG. 3, the controller 46 is fixedly arranged in the accommodating chamber 101 of the housing 10, while the temperature setting device 48 on the housing 10. The temperature setting device 48 includes a temperature-raising button 481 and a temperature-lowering button 482. The controller 46 is electrically connected with the temperature setting device 48 and the temperature sensor 42. The temperature setting device 48 can set a target temperature value, while the controller 46 can compare the target temperature value with the temperature detected by the temperature sensor 42 so as to control at least one of the following group: On/Off of the pump 20 and On/Off of the cooling chip 25. In other words, the controller 46 can control the pump 20 whether to run continuously so as to deliver or not to deliver the water in the cooling head 30 to the bag 40; or alternatively, to control the cooling chip 25 whether to run continuously so as to absorb or not to absorb the heat from the cooling head 30 and to make the water in the bag 40 comply with the target temperature value.

Further, according to the present invention, the temperature sensor 42 is fixed on the cooling head 30 so as to detect the temperature of the water at that location and to provide water temperature information for the user. The temperature display 44 is fixed on the housing 10 so as to display the water temperature being detected. In the present invention, the temperature display 44 relates to a liquid crystal display (LCD).

In other words, according to the present invention, the controller 46 can convert 110V public alternating current into a 12V direct current so as to supply power to the pump 20, the cooling chip 25, and the cooling fan 38. As such, overall power consumption for the cold/hot therapy device 1, according to the present invention, is less, and energy can be saved.

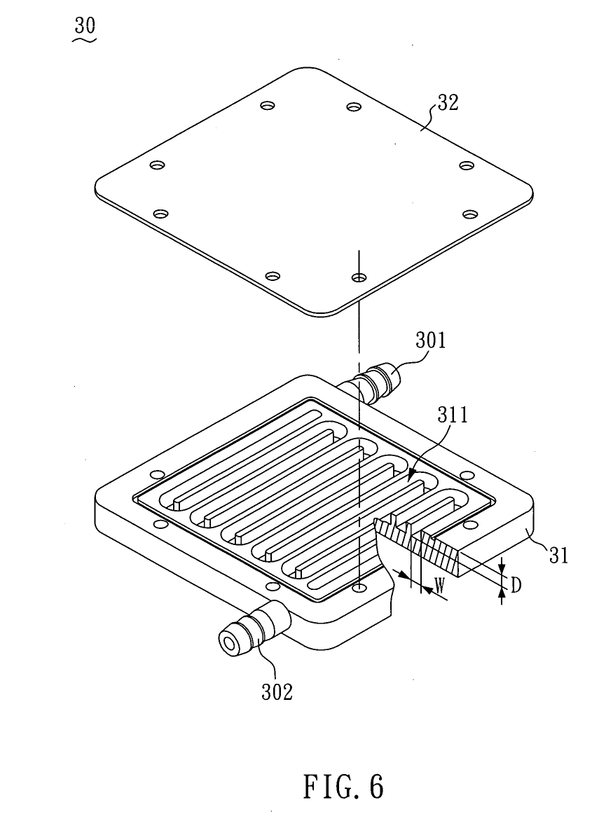

Now referring to FIG. 6, a perspective view illustrating a cooling head according to the present invention, and also to FIG. 3, the cooling head 30 includes a channel body 31 and a cooling surface 32. The channel body 31 is constituted, in series, by a plurality of channels 311 which are parallel from one another, wherein the depth D and the width W of the channel 311 are in a proportion of 3 to 4. The channel body 31 is formed zigzag so as to increase contact areas with the cooling surface 32, and as such, heat exchange effect can be enhanced, making water temperature reaches rapidly to a preset target value.

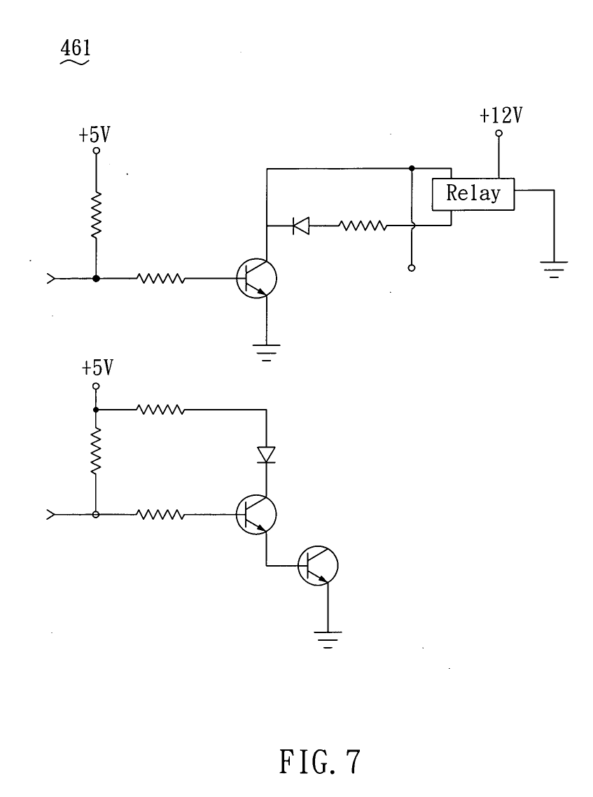

Further referring to FIG. 7, a diagram illustrating a reversible circuit according to the present invention, and also to FIG. 5, the controller 46 includes a reversible circuit 461 for controlling the cool-effecting surface 251 and the cooling surface 252 of the cooling chip 25 to become opposite effect, namely to provide a hot therapy function. That is to say, not only the present invention can provide a cold therapy function, but also the present invention can provide a hot therapy function. This can be achieved only by setting potential difference of pins so as to alter the cool-effecting surface 251 and the cooling surface 252 of the cooling chip 25, and to make the temperature of the bag 40 changed. It is advised that the cold therapy and the hot therapy range from 15° C. to 40° C., setting optionally by the user with the temperature setting device 48.

In view of the above, it is noted that the cold/hot therapy device 1 with temperature control, according to the present invention, can increase cooling effect of the cooling fan 38 by such structural arrangement as the hollow passage 102 passes through the housing 10 transversely, enhancing the effect of the cooling chip 25 and making the overall cold/hot therapy device 1 a more compact dimension and energy saving.

Although the present invention has been explained in relation to its preferred embodiments, it is to be understood that many other possible modifications and variations can be made without departing from the scope of the invention as hereinafter claimed.

What is claimed is:

1. A cold/hot therapy device with temperature control, comprising:
a housing, including an accommodating chamber and a hollow passage;
a pump, being fixedly arranged in the accommodating chamber of the housing, and including a pump inlet and a pump outlet;
a cooling chip, being fixedly arranged in the accommodating chamber of the housing, and including a cool-effecting surface and a cooling surface;
a cooling head, being fixedly arranged in the accommodating chamber of the housing, and being adjacent to the cool-effecting surface of the cooling chip, and including a water inlet and a water outlet, where the water inlet communicates with the pump outlet;
a cooling fin, being fixedly arranged on the cooling surface of the cooling chip, and being positioned in the hollow passage of the housing; and
a bag, including an inlet tube and an outlet tube, where the inlet tube communicates with the water outlet of the cooling head, and the outlet tube communicates with the pump inlet of the pump.

2. The cold/hot therapy device with temperature control as claimed in claim 1, further comprising a cooling fan which is fixedly arranged in the hollow passage and faces toward the cooling fin.

3. The cold/hot therapy device with temperature control as claimed in claim 1, further comprising a temperature sensor which is fixed on the cooling head.

4. The cold/hot therapy device with temperature control as claimed in claim 3, further comprising a temperature display, which is fixed on the housing, for displaying the water temperature being detected.

5. The cold/hot therapy device with temperature control as claimed in claim 3, further comprising a controller, fixedly arranged in the accommodating chamber of the housing, and a temperature setting device, fixedly arranged on the housing, wherein the controller is electrically connected with the temperature setting device and the temperature sensor; and wherein the temperature setting device can set a target temperature value, while the controller can compare the target temperature value with the temperature detected by the temperature sensor so as to control at least one of the following group: On/Off of the pump and On/Off of the cooling chip.

6. The cold/hot therapy device with temperature control as claimed in claim 5, wherein the controller further includes a reversible circuit for controlling the cooling chip so as to provide a cold or hot therapy function.

7. The cold/hot therapy device with temperature control as claimed in claim 1, wherein the cooling head includes a channel body and a cooling surface, wherein the channel body is constituted, in series, by a plurality of channels which are parallel from one another, and wherein the depth and the width of the channel are in a proportion of 3 to 4.

8. The cold/hot therapy device with temperature control as claimed in claim 1, wherein the housing consists of a base and a cover, the cover has a plurality of engaging tabs each having a through hole; while the base has a plurality of screw holes corresponding to the through holes.

9. The cold/hot therapy device with temperature control as claimed in claim 8, wherein the cover is provided with a handle such that the cold/hot therapy device can be portable and is easy to be carried.

10. The cold/hot therapy device with temperature control as claimed in claim 1, wherein the hollow passage passes transversely through the housing.
